(19) United States
(12) Patent Application Publication
(10) Pub. No.: US 2022/0194329 A1
(43) Pub. Date: Jun. 23, 2022

(54) Cart Sanitation Station

(71) Applicants: Shawnna Krull, Yuma, AZ (US); Brandy Bowman, Yuma, AZ (US)

(72) Inventors: Shawnna Krull, Yuma, AZ (US); Brandy Bowman, Yuma, AZ (US)

(21) Appl. No.: 17/203,937

(22) Filed: Mar. 17, 2021

Related U.S. Application Data
(60) Provisional application No. 63/126,816, filed on Dec. 17, 2020.

(51) Int. Cl. B60S 3/04; A61L 2/18; B08B 3/02; A61L 2/10

(52) U.S. Cl. CPC B60S 3/04; A61L 2/10; B08B 3/02; A61L 2/18

(57) ABSTRACT

The present invention relates generally to the field of sanitation equipment. More specifically, the present invention relates to a shopping cart sanitation system that sanitizes shopping carts, baskets and bins that pass therethrough. The system is comprised of a tunnel-like enclosure that is comprised of a sanitization chamber and a drying chamber, wherein the sanitization chamber sanitizes carts via a spray nozzle system that sprays disinfectant as well as a plurality of UV disinfecting lights. The drying chamber dries the carts, baskets and bins via a plurality of fans before the carts exit the station and are ready for use. In one embodiment, the system may further comprise an antimicrobial station.

### CROSS-REFERENCE TO RELATED APPLICATION

The present application claims priority to, and the benefit of, U.S. Provisional Application No. 63/126,816, which was filed on Dec. 17, 2020 and is incorporated herein by reference in its entirety.

### FIELD OF INVENTION

The present invention relates generally to the field of sanitation equipment. More specifically, the present invention relates to a shopping cart sanitation station that sanitizes shopping carts that pass through the station via a conveyor belt. The station is comprised of a tunnel-like enclosure having a both a sanitization chamber and a drying chamber, wherein the sanitization chamber sanitizes the carts via a spray nozzle system that sprays a disinfectant and/or santizies the carts via a plurality of UV disinfecting lights. The drying chamber dries the sanitized carts via a plurality of fans before the carts exit the station and are ready for use. In a further embodiment, the system may also comprise an additional station that sanitizes related and smaller items, such as baskets, bins, etc. Accordingly, the present disclosure makes specific reference thereto. Nonetheless, it is to be appreciated that aspects of the present invention are also equally applicable to other like applications, devices and methods of manufacture.

### BACKGROUND

Various germs, bacteria, pathogens, viruses, microbes, etc. are present on most public surfaces and can easily be transmitted amongst individuals by touching said surfaces, thereby facilitating the spread of potentially dangerous infectious diseases. Further, in the time of a pandemic such as COVID-19, there is a high risk of infection transmission in public places and via public surfaces. Establishments such as shopping malls, supermarkets, grocery stores and the like are frequently visited by both infected and healthy individuals, wherein the infected individuals may spread germs, bacteria, pathogens, viruses, etc. to the healthy individuals through contact or commonly used items and surfaces.

For example, in supermarkets, grocery stores, shopping centers, airports and other establishments, shopping carts, baskets and/or bins are typically available for use by customers while shopping or otherwise frequenting the establishment. Such shopping carts, baskets and bins may hold and transport food, clothing and other consumer items for purchase or the user's own personal belongings, such as a coat, hat, scarf, etc., wherein each of the items placed in the cart come into contact with the surfaces of the cart as does that user's hands when pushing or otherwise handling the cart. Therefore, such shopping carts, baskets, bins and other store items are highly susceptible to the spread of germs, bacteria, pathogens, viruses, etc. due to high traffic use among various individuals.

More specifically, users may not always wash their hands before touching the cart or items placed therein, which means that frequently touched surfaces on the carts and said items could become contaminated with germs, bacteria, pathogens, viruses, etc. from the user. Further, if not santizied frequently and properly, the shopping carts, baskets and bins may become unsanitary, dirty, and can easily spread germs, bacteria, pathogens, viruses, etc. via their surfaces. Consequently, the next individual to use the cart/basket/bin is then at risk for contracting and further spreading the germs, bacteria, pathogens, viruses, etc. of the infected individual that was transmitted via the cart to subsequent users.

In an effort to combat this issue, some merchants will manually and periodically sanitize their shopping carts, baskets and bins, or make sanitary wipes available to the customer for the customer to do so. However, this method of sanitizing carts suffers from a number of inherent limitations. First, manually sanitizing each cart after each use is both time consuming and highly inefficient, and it also requires the merchant to track each use of each cart, which is impractical. Second, because the presence of germs, bacteria, pathogens, viruses, etc. on the carts is not visible, there is no way of definitively knowing if the entire cart has been properly sanitized or if certain portions of the cart were overlooked. As such, missed spots or a non-thorough cleaning effort may not eliminate all of the germs, bacteria, pathogens, viruses, etc. on the cart, basket or bin surfaces (e.g. such as the interior of the cart/basket/bin). Additionally, making sanitary wipes available for customers to use assumes that they will in fact use the same and properly clean the cart, which is unrealistic and, as noted above, inefficient and largely ineffective.

Therefore, there still exists a potential risk for germs, bacteria, pathogens, viruses, etc. to spread even after the shopping carts, baskets and bins have been supposedly manually cleaned/disinfected. Further, frequent manual cleaning of each shopping cart, basket, and/or bin is a frustrating and tiresome process, and requires a substantial amount of effort and labor on the part of the merchant's employees. In addition, in the time of a pandemic, there is a higher demand for additional employees to attend to sanitizing measures, thereby resulting in elevated labor costs for businesses, who as a result may be required to hire additional employees simply for the purpose of cart sanitization. Additionally, manual cleaning typically involves the use of single use paper goods and sanitizing wipes (so as to not cross-contaminate surfaces), which increases expense and generates more waste.

Therefore, a long felt need in the art exists for a sanitation system that automatically sanitizes shopping carts, baskets, bins and other like items. There is also a long felt need in the art for a shopping cart sanitation system or station that thoroughly cleans and adequately sanitizes all surfaces of the shopping cart, basket and/or bin to prevent the spread of infectious germs, bacteria, pathogens, viruses, etc. Additionally, there is a long felt need in the art for a shopping cart sanitation station that does not require a significant amount of labor or employees to operate, and that prevents excessive environmental waste via the use of single use paper goods and wipes. Further, there is a long felt need in the art for a shopping cart sanitation station which effectively and efficiently sanitizes shopping carts, baskets and bins after each use, and provides a dry, sanitized cart, basket or bin for every new customer to use in a relatively short period of time. Finally, there is a long felt need in the art for shopping cart sanitation system that is relatively inexpensive to manufacture and maintain, and that is both safe and easy to use.

The subject matter disclosed and claimed herein, in one embodiment thereof, comprises a shopping cart sanitization system for sanitizing and drying shopping carts, baskets and bins. The system is comprised of a tunnel-like enclosure that is comprised of a sanitization chamber that chemically sanitizes and/or uses UV light to sanitize the shopping carts, a drying chamber that dries the sanitized carts and a conveyer belt system that propels the shopping carts, baskets and/or bins through the sanitation system. The interior of the sanitization chamber is further comprised of both a sanitizer/disinfectant sprayer and an ultraviolet light disinfection means, each of which is automatically activated when a shopping cart, basket or bin enters the sanitization chamber. Likewise, the drying chamber is comprised of a plurality of drying fans and blowers, each of which is automatically activated when a shopping cart, basket or bin enters the drying chamber from the sanitation chamber. Further, the sanitization chamber and the drying chamber are separated by a repositionable barrier to prevent the loss of sanitizing solution, and to create confined and separate sanitizing and drying areas of stations.

In this manner, the novel shopping cart sanitation system of the present invention accomplishes all of the forgoing objectives, and provides a relatively convenient, efficient and cost-effective solution to sanitizing and disinfecting a plurality of shopping carts, baskets and/or bins which may be frequently used by individuals in a retail/business setting to prevent and/or minimize the potential for the spread of infectious germs, bacteria, pathogens, viruses, etc. amongst individuals. The shopping cart sanitation system of the present invention is also autonomous, less expensive and more reliable than traditional manual cleaning methods and systems.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a shopping cart sanitation system. The sanitation system is comprised of a stainless steel tunnel-like enclosure that allows shopping carts to enter through a front continuous opening. The carts then pass an activation sensor via a conveyor belt through a sanitization chamber, wherein the carts are chemically sanitized with a sanitizing/disinfectant solution via a plurality of spray nozzles that spray a sanitizer and/or disinfectant agent on every surface of the cart. As an added measure, the sanitization chamber may further comprise a UV light disinfection system. Once the carts pass through the sanitization chamber, the conveyor system propels the carts through a repositionable barrier and into a drying chamber where the carts are properly dried before being returned to productive use by customers/patrons. The drying chamber is comprised of a plurality of air dryers and fans that blow air into the chamber to dry the sanitized carts via multiple vents. As previously stated, the sanitization chamber and the drying chamber are preferably separated by a repositionable strip curtain or barrier to prevent the loss of sanitizing solution and to create separate and confined sanitization and drying areas. Further, the system may further comprise a controller that allows a user to control the automatic operation of the nozzles, UV lights, conveyer belt, fans and other various sensors that may allow for the automatic operation of each respective chamber of the system.

In a further embodiment of the present invention, the shopping cart sanitization system may be comprised of a smaller conveyor belt system for passing smaller items, such as shopping baskets, bins or the like, through each of the sanitization and drying chambers so that the same do not interfere with the sanitization of the shopping carts. The basket/bin sanitizing conveyor station is identical in architecture to the shopping cart conveyor system and may also be comprised of its own stainless steel tunnel-like enclosure that allows baskets/bins to enter through a front continuous opening, if the baskets and bins do not pass through the same tunnel-like enclosure through which the shopping carts are propelled. More specifically, the baskets and bins are propelled via a conveyor belt through a sanitization chamber, which chemically sanitizes the baskets and bins with a sanitizing/disinfectant solution, and a drying chamber that dries the baskets and bins before they exit the system via a rear continuous opening to be returned to productive use by customers. As with the shopping cart sanitization system, the sanitization chamber for the baskets and bins may be comprised of a plurality of spray nozzles that spray a sanitizer/disinfectant agent on every surface of the baskets and bins and a UV light disinfection system for further disinfecting of the same. The drying chamber is also comprised of a plurality of air dryers and fans that blow air into the chamber to dry the sanitized baskets and bins via a plurality of vents. The sanitization chamber and the drying chamber are preferably separated by a repositionable barrier to prevent the loss of sanitizing solution, and to create a confined sanitization and drying area. Further, a controller (manned or unmanned) may control the operation of the basket/bin sanitization station in the same manner as described above, and the two sanitization systems, if separated by one another, may still share many of the same components such as, but not limited to, the controller, sanitizing solution tank, power supply, etc. In this manner, a user may separately and simultaneously sanitize both a plurality of shopping carts and shopping baskets/bins.

In a further embodiment of the present invention, the shopping cart and/or basket and bin sanitizing systems may further comprise a means for applying an anti-microbial coating to the sanitized carts before the same are returned to productive use. The anti-microbial agent will act to kill various infectious germs, bacteria, pathogens, microbes, viruses, etc. on contact even after the shopping carts, baskets and bins have been returned to productive use, and the anti-microbial agent may be applied to the shopping carts, baskets and bins in the form of a fast-drying mist in its own separate chamber or as part of the drying chamber. In this manner, the sanitized shopping carts, baskets and bins will be effective at stopping, or at least slowing down, the spread of harmful infectious germs, bacteria, pathogens, microbes, viruses, etc. even after the same have been returned to productive use.

In yet another embodiment of the present invention, a method of quickly and efficiently sanitizing and drying a plurality of shopping carts, baskets and bins is disclosed. The method is comprised of the initial step of placing one or more carts/bins at the front opening of a cart sanitization system and on a conveyor. Next, the conveyor is activated by an activation sensor to transport the carts/bins through a sanitization chamber, wherein a sanitizing solution is sprayed on the carts/bins via nozzles and/or an ultraviolet disinfecting light is automatically activated to shine on the carts and bins as they move across the sanitizing chamber. Next, the conveyer belt carries the carts and/or bins through a repositionable barrier and into a drying chamber, wherein the carts and bins are blown dry via a plurality of blowers and/or air dryers. Finally, the freshly sanitized carts and/or bins exit the drying chamber via a rear egress opening and are ready to be returned to productive use. In an alternative embodiment, the sanitized carts and/or bins may further pass through an antimicrobial station on the conveyor, wherein the carts and bins are misted with a fast drying antimicrobial agent.

As such, the shopping cart sanitization system of the present invention provides merchants and other business owners with a hands-free system for quickly, efficiently and thoroughly sanitizing shopping carts, bins and/or baskets. The sanitization system also ensures adequate sanitization of all surfaces of the carts, baskets and bins via a plurality of high pressure disinfectant spray nozzles which spray disinfectant/sanitizer on the carts, baskets and bins as they pass through the sanitization chamber, wherein a plurality of UV disinfecting lights are also used to further sanitize the carts, bins and/or baskets of germs, bacteria, pathogens, viruses, etc., before they are dried in the drying chamber and returned to productive use.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a shopping cart sanitation system of the present invention in accordance with the disclosed architecture;

FIG. 2 illustrates a perspective view of one potential alternative embodiment of a shopping cart sanitation system of the present invention in accordance with the disclosed architecture, wherein the system has both a first conveyor system for sanitizing larger shopping carts and a second conveyor system for sanitizing smaller shopping receptacles, such as shopping baskets and/or bins;

FIG. 3 illustrates a side diagrammatic view of one potential embodiment of the shopping cart sanitation system of the present invention in accordance with the disclosed architecture, wherein the system is comprised of a sanitization chamber, a drying chamber and an antimicrobial chamber;

FIG. 4 illustrates a side diagrammatic view of one potential embodiment of the sanitization chamber of the shopping cart sanitation system of the present invention in accordance with the disclosed architecture, wherein a plurality of shopping carts are entering the sanitization chamber on an automated conveyor; and

FIG. 5 illustrates a side diagrammatic view of one potential embodiment of the drying chamber of the shopping cart sanitation system of the present invention in accordance with the disclosed architecture, wherein a plurality of shopping carts are passing through the drying chamber on an automated conveyor and are about to exit the egress.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

The present invention, in one exemplary embodiment, is comprised of a shopping cart sanitation system that is comprised of a stainless steel tunnel-like enclosure having a conveyor, an ingress, an egress and a continuous pathway extending between the ingress and the egress, wherein a plurality of shopping carts, bins or baskets may be propelled through the pathway on the conveyor. The pathway further comprises a sanitization chamber, a drying chamber and an optional antimicrobial chamber. In the sanitization chamber, the shopping carts, baskets and/or bins are chemically sanitized with a sanitizing/disinfectant solution that is sprayed through a plurality of spray nozzles positioned around the conveyor, each of which is in fluid communication with a tank holding a supply of sanitizing and/or disinfecting solution. While in the sanitization chamber, the carts, baskets and bins may also be subjected to a plurality of UV disinfecting lights positioned around the conveyor to further sanitize the carts, baskets and bins.

Once properly sanitized, the conveyor transports the carts, baskets and bins through a repositionable barrier and into the drying chamber. In the drying chamber, the carts, baskets and bins are dried via a plurality of fans or blowers positioned around the conveyor and the same then exit the drying chamber and the enclosure itself, wherein the properly and thoroughly sanitized carts, baskets and bins are ready to be returned to productive use. Alternatively, the carts, baskets and bins may first be subjected to an antimicrobial chamber wherein the carts, baskets and bins are misted with a fast drying antimicrobial solution that is sprayed through a plurality of misting nozzles positioned around the conveyor, each of which is in fluid communication with a tank holding a supply of the antimicrobial solution before exiting the disclosure.

Additionally, the cart sanitization system also comprises a controller station that allows a user to control the automatic operation of the various components of the cart sanitization system including, without limitation, the nozzles, UV lights, conveyer belt, fans, blowers, as well as a plurality of sensors for sensing the position various sensors that may allow for the automatic operation of each chamber.

A differing embodiment of the shopping cart sanitization may also be comprised of a smaller basket/bin sanitizing system that may comprise the top surface of the shopping cart sanitization system or, alternatively, either side surface. The basket/bin sanitizing system is identical in architecture to the shopping cart system and is also comprised of a stainless steel tunnel-like enclosure that allows baskets/bins to enter the same through a front continuous opening. The baskets/bins then pass via a conveyor belt through a sanitization chamber which chemically sanitizes the baskets/bins with a sanitizing solution, and a drying chamber that dries the baskets/bins before the carts exit the system via a rear continuous opening to be returned to productive use by customers. The sanitization chamber may be comprised of a plurality of spray nozzles that spray sanitizer on every surface of the baskets/bins, and/or a UV light disinfection system. The drying chamber may be comprised of a plurality of air dryers and fans that blow air into the chamber to dry the sanitized baskets/bins via a plurality of vents. The sanitization chamber and the drying chamber are preferably separated by a strip curtain to prevent the loss of sanitizing solution, and to create a confined sanitization and drying area. Further, the system may further comprise an antimicrobial station as described above, and a controller station that allows a user to control the automatic operation of the nozzles, UV lights, conveyer belt, fans and other various sensors that may allow for the automatic operation of each chamber. However, as noted above, it is contemplated that both the shopping cart sanitization system and the basket/bin sanitization system may share several components (e.g. a controller, a disinfectant tank, etc.).

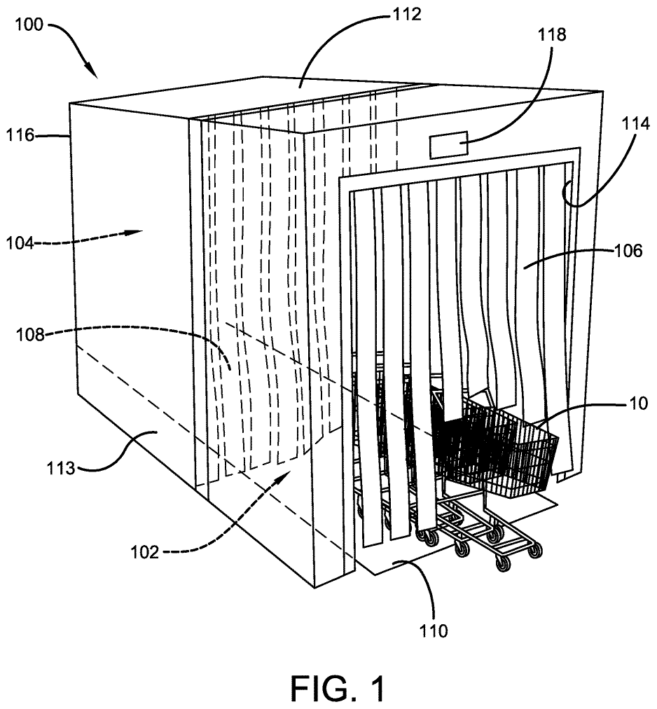

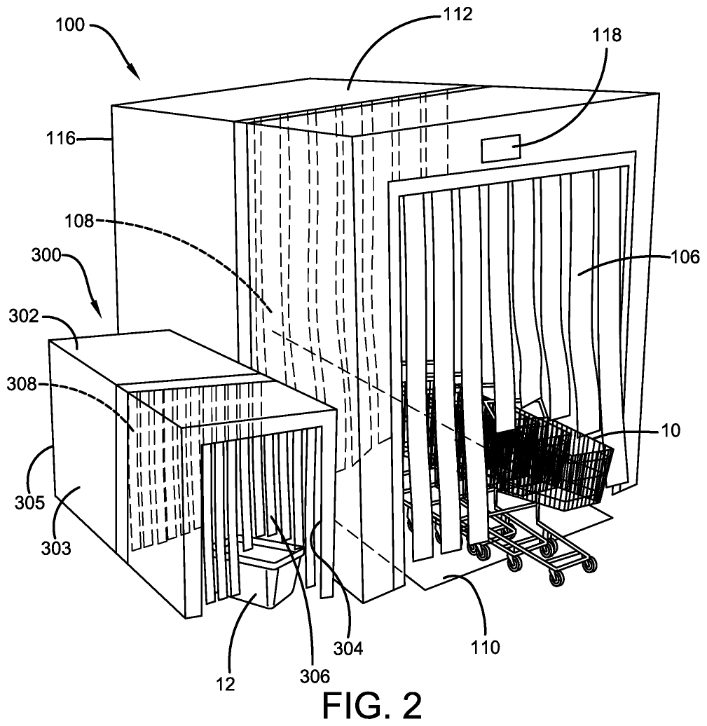

Referring initially to the drawings, FIG. 1 illustrates a perspective view of one potential embodiment of a shopping cart sanitation system 100 of the present invention in accordance with the disclosed architecture, and FIG. 2 illustrates a perspective view of one potential alternative embodiment of a shopping cart sanitation system 100 positioned adjacent to a basket/bin sanitization system 300. It should be noted that the basket/bin sanitation station 300 is substantially identical in structure, performance, components, form and function to the shopping cart sanitization system 100, other than the fact that it is physically smaller. As such, the remainder of the detailed description describes both the shopping cart sanitation system 100 and the basket/bin sanitation system 300 unless explicitly stated otherwise, and any reference to the shopping cart sanitizing system 100 also applies and describes the basket/bin sanitization system 300.

The system 100 is comprised of a tunnel-like or rectangular-like enclosure that is comprised of a top surface 112, two generally parallel side surfaces 113, a continuous front opening 116 through which shopping carts 10 can enter the system 100 via a conveyor belt 110 or grooved track (not shown) that secures and guides the movements of the carts 10 and a continuous rear opening 114 wherein shopping carts 10 can exit the system 100 via the conveyor belt 110 or grooved track (not shown). The conveyor belt 110 effectively comprises the bottom surface of the station 100, but a differing embodiment of the system 100 may be further comprised of an additional bottom surface (not shown) positioned below the conveyor belt 110 that is comprised of the same materials as the top and side surfaces 112, 113, and/or a plurality of removable, locking wheels that allow the entire system 100 to move easily from one location to the other. Nonetheless, in differing embodiments, the system 100 may also be in the general shape of any of a plurality of three-dimensional shapes, such as triangular, oval, oblong, circular, half-circular, etc., and may be comprised of a plurality of durable materials such as, but not limited to, stainless steel, steel, aluminum, fiberglass, plastic, etc. Further, the outer surface of the sanitization system 100 may be comprised of a plurality of LED or non-led display panels that allows a business to display a logo or advertisements and provide outside lighting to the system 100.

Each opening 114, 116 is further comprised of a plurality of strip curtains 106 that are comprised of vinyl or other suitable material such that the curtains 106 serve to contain disinfectant and other overspray within the system 100, preventing said overspray from saturating the surrounding environment. Above each opening 114, 116, the system 100 may also be comprised of a sensor 118 that detects the presence (e.g. at the front opening 116) and/or the absence (e.g. at the rear opening 114) of shopping carts 10 within the system 100 to automatically activate or deactivate the sanitization/drying stations, and to automatically start the movement of the conveyor belt 110 when the presence of one or more shopping carts 10 is detected near or inside the front opening 116. Similarly, the sensor 118 above the rear opening 114 can detect the presence or absence of the shopping carts 10 to effectively start or stop the conveyor belt 110 and drying system when all carts 10 have exited the system 100. Therefore, to allow a cart 10 or a series of carts 10 to travel through the system 100, a user needs only to push the cart(s) 10 into the front opening 116 of the system 100 where their presence will be detected by the sensor 118, and the system 100 will then automatically activate or deactivate the system accordingly as the carts 10 move throughout each station or chamber (as described more fully below) until they ultimately exit the system 100 at egress 114.

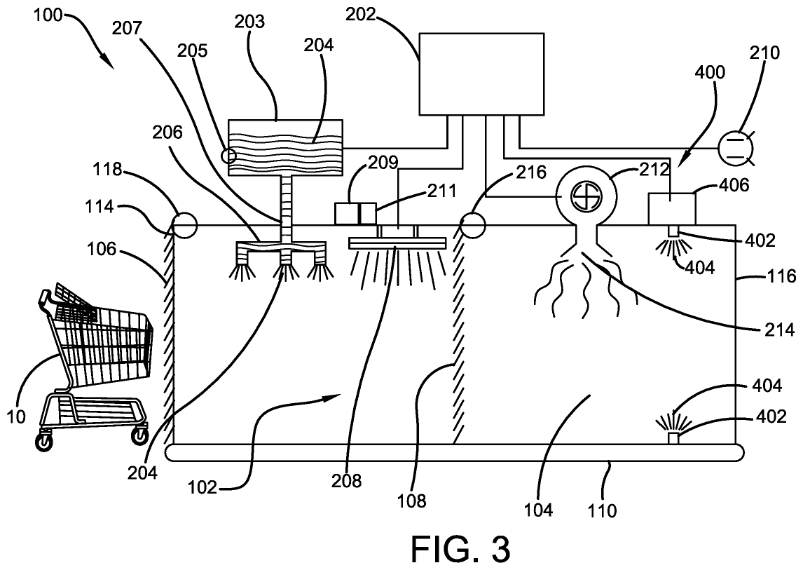

FIG. 3 illustrates a side diagrammatic view of one potential embodiment of the shopping cart sanitation system 100 of the present invention in accordance with the disclosed architecture, wherein the system is comprised of a sanitization chamber 102, a drying chamber 104 and an antimicrobial station or chamber 400. More specifically, the interior of the system 100 is generally divided into two or three stations, areas or chambers, which are further divided by a repositionable barrier or middle strip curtain 108 that is comprised of vinyl or other suitable material such that the curtains 108 serve to contain disinfectant and other overspray within the sanitization chamber 102, which is defined as the internal space positioned between the front opening 116 and the drying chamber 104.

The sanitization chamber 102 is comprised of a plurality of high-pressure spray nozzles 206 preferably positioned on the system's interior top, bottom, and/or side surfaces (i.e., positioned around the conveyor 110), and spray liquid disinfectant in a droplet or mist form onto all surfaces of the shopping carts 10 as they pass through the sanitization chamber 102 on conveyor belt 110, as best seen in FIG. 3. Further, the top surface 112 of the system 100 or the interior top surface may further be comprised of at least one sanitizer solution tank 203 that houses a liquid disinfectant 204 and supplies the same to the various spray nozzles 206 via a tubing system 207. In the preferred embodiment of the station 100, the tank 203 is further comprised of a monitoring sensor 205 that monitors the solution levels in the tank 203, and a troubleshooting sensor (not shown) that detects any mechanical errors within the system 100. Notifications regarding the level of available solution 204 within the tank 203, troubleshooting or any other user specified condition can be provided to a user through a visible and/or audible alarm system 209 that may comprise a plurality of lights and speakers (not shown) on the interior and/or exterior of the system 100. Additionally, the notifications may be transmitted to a smart device via a wireless communication module 211 that is linked to a mobile application.

The cart detection sensor 118 may also serve as a cart capacity sensor that provides a visual or audible indication to the user of the number of carts 10 within the system at any one time, or if the carts are jammed in some capacity. The indication may be in the form or a display, an audible alarm or an alert via a push notification via the mobile application. Further, a user may similarly be alerted if the number of carts 10 within the system 100 exceeds the capacity of the system 100.

In addition, the mobile application may also allow a user to executively control one or more features of the system 100 such as, but not limited to, the spray nozzles 206, UV light system 208, air dryers/fans 212, on/off power, etc. Alternatively, the spray nozzles 206, UV lights 208, misting devices 402, etc., may be in electrical communication with, and controlled by, a physical control panel 202 in the form of a computer system or processor that, for example, receives signals from the sensor 118 at the front opening 116 and sends a signal to both the spray nozzles 206 and the UV lights 208 to start dispensing solution 204 and emitting UV light when carts 10 are detected within the station 100 in the sanitization chamber 102.

More specifically and as stated above, the sanitization chamber 102 may also additionally or alternatively be comprised of a plurality of ultraviolet disinfecting lights 208 positioned on the on the interior top, bottom, and/or side surfaces of the system 100 (i.e., positioned around the conveyor belt 110) to further sanitize the shopping carts 10 passing through the sanitization chamber by killing germs, bacteria, pathogens, viruses, etc. via the UV light. It is contemplated that as the conveyor belt 110 guides the shopping carts 10 through the sanitizing chamber 102, the nozzles 206 and UV lights 208 will be activated via sensor 118 of a separate, unique sensor (not shown). Alternatively, the nozzles 206 and UV lights 208 may be activated via a weight sensor (not shown) within the conveyor belt 110 that only activates the nozzles 206 and UV lights 208 when a sufficient amount of weight is detected on the conveyor belt 110 (e.g. the weight of at least one shopping cart 10).

As stated above, the system 100 may comprise a plurality of external controls on the control panel 202 positioned on the interior and/or exterior of the system 100 to activate or deactivate the nozzles 206, UV lights 208, etc. manually. In yet another embodiment, each of the nozzles 206 and the UV lights 208 may be in electrical communication with a plurality of intensity level settings on the control panel 202 such as, but not limited to, a “light” cleaning level, a “medium” cleaning level and a “heavy” cleaning level, wherein each level is configured to be set through the controller 202 or mobile application to allow the user to select the intensity at which the nozzles 206 and UV lights 208 operate. Th system 100 may also comprise a number of indicators such as, but not limited to, a solution level indicator and a “system needs attention” indicator.

After the sanitization of the shopping carts 10 is complete, the carts 10 travel through the middle strip curtain 108 via the conveyor belt 110 to the drying chamber 104, which is present between the middle strip curtain 108, which separates the drying chamber 104 from the sanitization chamber 102, and the rear opening 114. The drying chamber 104 is comprised of a plurality of air dryers or blowers 212 (e.g. fans) and/or heat lamps (not shown) that may be positioned along the interior top, bottom, and/or side surfaces of the drying chamber 104 (i.e., around the conveyor belt 110) and that dispel a flow of air into the drying chamber 104 via a plurality of vents 214 to dry the moisture present on the shopping carts 10 as best seen in FIG. 3.

After the sanitization and drying of the shopping carts 10 is complete, in one embodiment hereof, the carts 10 may further travel through an antimicrobial station 400. The antimicrobial station 400 is comprised of a plurality of sprayers or misters 402 that coat the entire surface of the shopping carts 10 with a fast drying antimicrobial solution 404. More specifically, the antimicrobial solution 404 is stored in an antimicrobial solution holding tank 406 until needed, wherein the holding tank 406 is in fluid communication with each of the plurality of sprayers or misters 402.

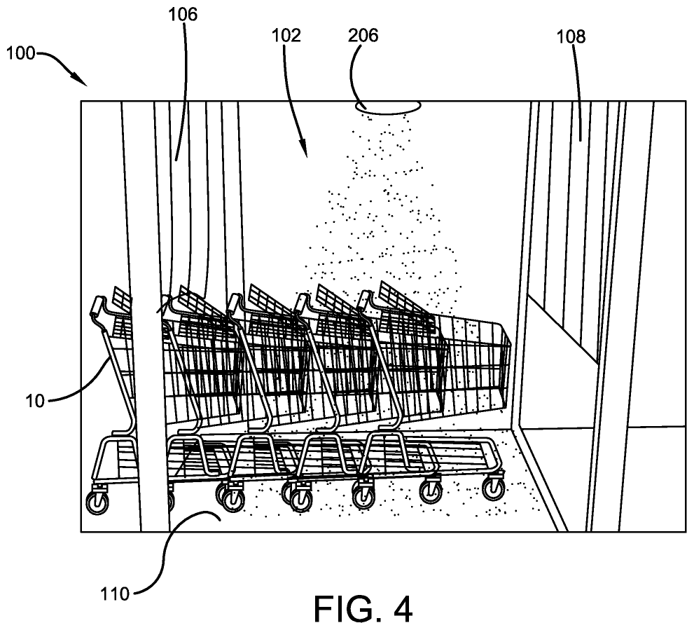

FIG. 4 illustrates a side diagrammatic view of one potential embodiment of the sanitization chamber 102 of the shopping cart sanitation system 100 of the present invention in accordance with the disclosed architecture, wherein a plurality of shopping carts 10 are entering the sanitization chamber 102 on an automated conveyor 110, wherein the same will be sprayed with the sanitizing solution before moving on to the drying chamber 104. Like the spray nozzles 206 and UV lights 208 of the sanitization chamber 102, the air dryers/fans 212 may be activated when the carts 10 pass through the midpoint strip curtain 108 after being detected by a presence sensor 216 located at the midpoint, or alternatively by a fixed weight sensor (not shown) that may be positioned along the conveyor belt 110 pathway near the midpoint, and which activates the air dryers/fans 212 when the weight of at least one cart 10 is detected at the midpoint area. Preferably, the presence sensor 216 and/or weight sensor are also in electrical communication with the controller 202, which receives signals from the sensors 216 to activate the fans 212. It is further contemplated that the controller 202 is in electrical communication with all other sensors 118 and other components of the system 100 to provide an automatic and effective mechanism of sanitizing shopping carts, basket, bins and other shopping receptacles.

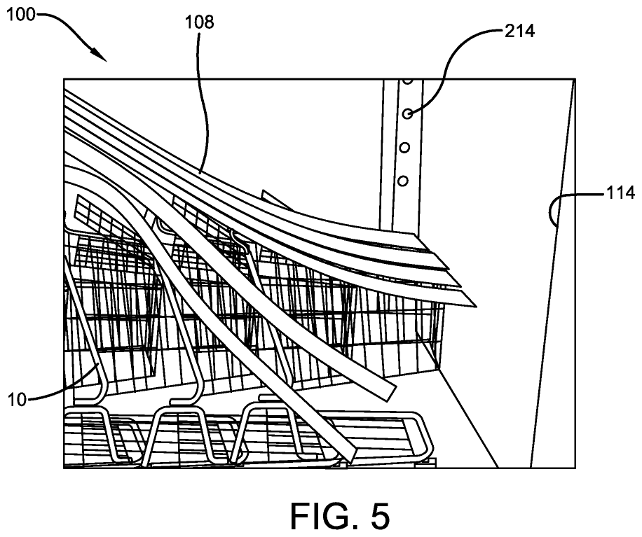

FIG. 5 illustrates a side diagrammatic view of one potential embodiment of the drying chamber 104 of the shopping cart sanitation system 100 of the present invention in accordance with the disclosed architecture, wherein a plurality of shopping carts 10 are passing through the drying chamber 104 on an automated conveyor 110 and are about to exit the egress 114. More specifically, after the sensor 118 positioned at the rear opening 114 and/or the weight sensor (not shown) in the conveyor belt 110 no longer detects the presence of any carts 10 within the drying area 104, the operation of the fans 212 and heating elements may be stopped to ensure the carts 10 are dried in minimum time with no moisture left on their surface and in an energy efficient manner. The sanitized and dried shopping carts 10 then exit the system 100 (assuming the optional antimicrobial station 400 is not present) through the rear opening 114 via the conveyer belt 110, wherein the dried and sanitized carts 10 may be returned to productive use by customers.

As noted above, FIG. 2 illustrates a perspective view of another potential embodiment of a shopping cart sanitation station 100 of the present invention that is further comprised of a basket/bin sanitation station 300 in accordance with the disclosed architecture. In this embodiment, the basket/bin station 300 may be positioned alongside the shopping cart sanitization system 100, although in differing embodiments of the cart station 10, the basket/bin station 300 may also be positioned above or below the system 100, or may be a stand-alone device. As also noted above, the basket/bin sanitization system 300 may be comprised of the exact same components as the cart sanitization system 100, and functions in substantially, if not the exact, same manner. As such, the basket/bin sanitization station 300 may comprise a conveyor belt 301, a top surface 302, two generally parallel side surfaces 303, a front/rear opening 305/304 that is further comprised of a strip curtain 306 and a strip curtain at the midpoint 308 of the station 300 body.

Although it is contemplated that the basket/bin station 300 is comprised of its own controller (not shown), it may be controlled by the controller 202 of the cart sanitization system 100, and/or by the same mobile application that can be used to control the system 100. Similarly, the same sanitization tank 203 may be used by both the systems 100, 300 although the basket/bin station 300 is preferably comprised of its own tank (not shown). In differing embodiments of the systems, the shopping cart sanitization system 100 and/or the basket/bin sanitization system 300 may be battery operated, solar-powered and/or powered by an electrical power source 210.

The cart sanitization system 100 and basket/bin sanitation system 300 can operate simultaneously based on consumer demand and requirements to accommodate any size and shape of shopping cart 10 or basket/bin 12 that may be used in retail shops, grocery stores, malls, airports, hospitals etc., and to thoroughly sanitize and dry each cart 10 and basket/bin 12 accordingly. Therefore, the sanitization systems 100, 300 reduce the spread of germs, viruses and bacteria on shopping carts, baskets, bins and any other shopping receptacle.

As noted above, another embodiment of the present invention may also be comprised of a method of sanitizing and drying shopping carts and/or baskets/bins or other devices of the like. The method is comprised of the steps of placing one or more carts/bins at the front opening of a sanitizing station, automatically moving the carts/bins through the station via conveyer belt and through a sanitizing chamber wherein sanitizing solution is sprayed on the carts/bins via nozzles and Ultraviolet disinfecting light is automatically activated to shine on the carts/bins as they move across the sanitizing chamber. Then, a conveyer belt carries the carts/bins to a drying chamber from the sanitizing region to dry the carts/bins via blowers and air dryers. Finally, the carts/bins may be coated with a fast drying antimicrobial solution before exiting the system via the rear exit opening.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “cart station”, “shopping cart sanitation system”, and “shopping cart system” are interchangeable and refer to the sanitation systems 100, 300 of the present invention.

Notwithstanding the forgoing, the sanitation systems 100, 300 of the present invention can be of any suitable size and configuration as is known in the art without affecting the overall concept of the invention, provided that it accomplishes the above stated objectives. One of ordinary skill in the art will appreciate that the size, configuration and material of the sanitation systems 100, 300 as shown in the FIGS. are for illustrative purposes only, and that many other sizes and shapes of the sanitation systems 100, 300 are well within the scope of the present disclosure. Although the dimensions of the sanitization systems 100 are important design parameters for user convenience, the sanitation systems 100, 300 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A shopping receptacle sanitization system comprising:
an enclosure;
a conveyor;
a sanitization station; and
a drying station.

2. The shopping receptacle sanitization system as recited in claim 1 further comprising an anti-microbial station.

3. The shopping receptacle sanitization system as recited in claim 1, wherein the enclosure is a tunnel like structure comprising an ingress, the sanitization station, the drying station and an egress.

4. The shopping receptacle sanitization system as recited in claim 3, wherein a repositionable barrier is positioned within the tunnel like structure between the sanitization station and the drying station.

5. The shopping receptacle sanitization system as recited in claim 4, wherein the conveyor extends between the ingress and the egress.

6. The shopping receptacle sanitization system as recited in claim 1 further comprising a control station.

7. The shopping receptacle sanitization system as recited in claim 1, wherein the sanitization station is further comprised of a spraying component and a UV disinfecting light component.

8. The shopping receptacle sanitization system as recited in claim 7, wherein the spraying component is comprised of a plurality of spraying nozzles in fluid communication with a tank holding a supply of a sanitizing agent.

9. The shopping receptacle sanitization system as recited in claim 8, wherein the plurality of spraying nozzles are positioned around the conveyor.

10. The shopping receptacle sanitization system as recited in claim 7, wherein the UV disinfecting light component is comprised of a plurality of UV disinfecting lights positioned around the conveyor.

11. The shopping receptacle sanitization system as recited in claim 1, wherein the drying station is comprised of a plurality of blowers positioned around the conveyor.

12. The shopping receptacle sanitization system as recited in claim 2, wherein the antimicrobial station is comprised of a plurality of misting nozzles in fluid communication with a tank holding a supply of an antimicrobial agent.

13. The shopping receptacle sanitization system as recited in claim 12, wherein the plurality of misting nozzles are positioned around the conveyor and between the drying station and an egress.

14. A shopping receptacle sanitization system comprising:
an enclosure having an ingress and an egress;
a first conveyor and a second conveyor, wherein each of the first and second conveyors extend between the ingress and egress;
a sanitization station;
a drying station;
an antimicrobial station; and
a control station.

15. The shopping receptacle sanitization system as recited in claim 14, wherein a repositionable barrier is positioned within the enclosure between the sanitization station and the drying station.

16. The shopping receptacle sanitization system as recited in claim 14, wherein the sanitization station is further comprised of (a) a spraying component comprised of a plurality of spraying nozzles positioned around each of the first and second conveyors and in fluid communication with a tank holding a supply of a sanitizing agent, and (b) a UV disinfecting light component comprised of a plurality of UV disinfecting lights positioned around each of the first and second conveyors.

17. The shopping receptacle sanitization system as recited in claim 14, wherein the drying station is comprised of a plurality of blowers positioned around each of the first and second conveyors, and the antimicrobial station is comprised of a plurality of misting nozzles positioned around each of the first and second conveyors and in fluid communication with a tank holding a supply of an antimicrobial agent.

18. A method of sanitizing a shopping receptacle comprising the steps of:
positioning a shopping receptacle on a conveyor at an ingress point of a sanitization system;
sensing the presence of the shopping receptacle and activating the conveyor;
spraying an entire surface of the shopping receptacle with a sanitizing solution as the conveyor transfers the shopping receptacle through a sanitization chamber;
exposing the entire surface of the shopping receptacle to a UV disinfecting light as the conveyor transfers the shopping receptacle through the sanitization chamber;
drying the entire surface of the shopping receptacle with a plurality of blowers as the conveyor transfers the shopping receptacle through a drying chamber;
spraying the entire surface of the shopping receptacle with an antimicrobial solution as the conveyor transfers the shopping receptacle towards an egress; and
sensing the presence of the shopping receptacle at the egress and stopping the conveyor.

19. The method of sanitizing a shopping receptacle as recited in claim 18, wherein a repositionable barrier is positioned between the sanitization chamber and the drying chamber.

20. The method of sanitizing a shopping receptacle as recited in claim 18, wherein the sanitization system further comprises a control panel, a sanitizing solution reservoir for storing the sanitizing solution and that is in fluid communication with a plurality of spray nozzles, a sanitizing solution reservoir for storing the sanitizing solution and that is in fluid communication with a plurality of spay nozzles, and an antimicrobial solution reservoir for storing the antimicrobial solution and that is in fluid communication with a plurality of sprayers.
